(19) United States
(12) Patent Application Publication
(10) Pub. No.: US 2025/0176893 A1
(43) Pub. Date: Jun. 5, 2025

(54) BIOLOGICAL INFORMATION DETECTION SYSTEM

(71) Applicant: Murata Manufacturing Co., Ltd., Kyoto (JP)

(72) Inventor: Hirofumi TSUCHIMOTO, Kyoto (JP)

(21) Appl. No.: 18/966,643

(22) Filed: Dec. 3, 2024

(30) Foreign Application Priority Data
Dec. 4, 2023 (JP) 2023-204708
Oct. 24, 2024 (JP) 2024-187149

(51) Int. Cl. A61B 5/302

(52) U.S. Cl. CPC A61B 5/302

(57) ABSTRACT

A biological information detection system includes a biosensor and a case that is capable of housing the biosensor. The biosensor includes a first storage unit and a first communication unit. The case includes a second communication unit, a third communication unit, and a case controller. The case controller is capable of performing an information reception process and an output process. The information reception process is a process of receiving biological information and sensor identification information by using the second communication unit. The output process is a process of transmitting, from the third communication unit to an external device, the biological information and the sensor identification information, which are received in the information reception process, in association with each other.

### CROSS REFERENCE TO RELATED APPLICATION

This application claims priority from Japanese Patent Application No. 2023-204708 filed on Dec. 4, 2023 and Japanese Patent Application No. 2024-187149 filed on Oct. 24, 2024. The contents of these applications are incorporated herein by reference in its entirety.

### BACKGROUND OF THE DISCLOSURE

### 1. Field of the Disclosure

The present disclosure relates to a biological information detection system.

### 2. Description of the Related Art

A test device described in Japanese Unexamined Patent Application Publication No. 2010-279701 is a device for blood glucose testing. The test device includes a container, a meter, and a housing of the meter. The container has a cylindrical shape in which one end opens and the other end is closed. The container is used to house a test media. The test media is attached to the meter in blood glucose testing. The meter is capable of testing the amount of glucose in a sample of whole blood applied to the test media. The housing of the meter is attachable to the container. In the state in which the housing is attached to the container, the housing closes the opening of the container. The test device is capable of detecting the state in which a test media has been inserted to the interface of the meter properly. When it is detected that a test media has been inserted to the interface properly, the meter is capable of determining the concentration in the sample applied to the test media. It is possible to transmit the data determined by the meter to an external device through an input/output unit of the meter.

### BRIEF SUMMARY OF THE DISCLOSURE

Multiple test devices as described in Japanese Unexamined Patent Application Publication No. 2010-279701 may be used in the same facility. In contrast, when the test device described in Japanese Unexamined Patent Application Publication No. 2010-279701 transmits, to an external device, data obtained by the meter, the data does not include information indicating which test device is used in measurement of the data. Therefore, when data is received from individual test devices, it is difficult to manage the multiple pieces of received data.

To solve the issue described above, the present disclosure provides a biological information detection system including a biosensor that detects biological information of a subject, and a case that is capable of housing the biosensor. The biosensor includes a first storage unit that stores the detected biological information and sensor identification information specific to the biosensor, and a first communication unit that transmits the biological information and the sensor identification information which are stored in the first storage unit. The case includes a second communication unit that receives the biological information and the sensor identification information which are transmitted by the first communication unit, a third communication unit that wirelessly transmits, to an external device, the biological information and the sensor identification information which are received by the second communication unit, and a case controller that controls communication through the second communication unit and communication through the third communication unit. The case controller is capable of performing an information reception process of receiving the biological information and the sensor identification information by using the second communication unit, and an output process of transmitting, from the third communication unit to the external device, the biological information and the sensor identification information which are received in the information reception process, in association with each other.

According to the configuration, the external device contains biological information and sensor identification information. Therefore, a user of the external device may confirm these pieces of information to manage which biosensor is used in measurement of the biological information.

### BRIEF DESCRIPTION OF THE SEVERAL VIEWS OF THE DRAWINGS

FIG. 1 is a diagram illustrating the appearance of a biological information detection system according to a first embodiment;

FIG. 2 is an enlarged view of an area near a sensor unit of a biosensor according to the first embodiment;

FIG. 3 is a schematic diagram illustrating the electrical configuration of a biosensor according to the first embodiment;

FIG. 4 is a diagram illustrating a case and a biosensor, which is housed in the case, according to the first embodiment;

FIG. 5 is a sequence chart for describing an exemplary process in a biological information detection system according to the first embodiment;

FIG. 6 is a perspective view of a biosensor according to a second embodiment;

FIG. 7 is a side view of a biological information detection system according to the second embodiment;

FIG. 8 is a diagram illustrating a case and a biosensor, which is housed in the case, according to a modified example; and

FIG. 9 is a sequence chart for describing an exemplary process in a biological information detection system according to the modified example.

### DETAILED DESCRIPTION OF THE DISCLOSURE

### First Embodiment

A biological information detection system according to a first embodiment will be described below by referring to the drawings. In the drawings, components may be enlarged for illustration to facilitate understanding. The dimensional ratios of components may be different from actual ones or those in different drawings.

About the Overall Configuration of a Biological Information Detection System

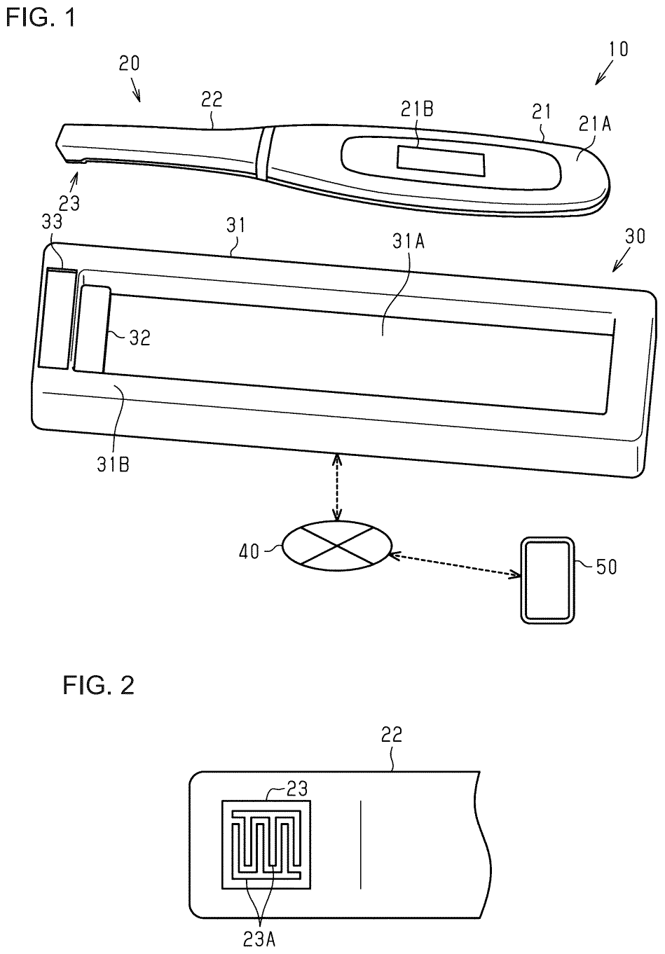

As illustrated in FIG. 1, a biological information detection system 10 includes a biosensor 20, a case 30 which is capable of housing the biosensor 20.

In the first embodiment, the biosensor 20 is a water-amount measurement device which detects, as biological information BI of a subject, the amount of water in their oral cavity. The biosensor 20 includes a body unit 21, an insertion unit 22, and a sensor unit 23.

The body unit 21 has an approximately rectangular parallelepiped shape elongating in one direction as a whole. The body unit 21 is a portion held by a user in use of the biosensor 20. The body unit 21 has a top surface 21A. In the first embodiment, the top surface 21A is a surface on one side, which is largest among the outer surfaces of the body unit 21. In the description below, the direction, in which the top surface 21A of the body unit 21 is oriented, is assumed to be the top side of the biosensor 20, and its opposite side is assumed to be the bottom side of the biosensor 20.

The body unit 21 includes a display screen 21B. The display screen 21B is exposed on the top surface 21A of the body unit 21. The display screen 21B is positioned approximately at the center in the longitudinal direction of the body unit 21. The display screen 21B is capable of displaying character data and the like which indicate the amount of water measured by the biosensor 20.

The insertion unit 22 has an approximately rectangular parallelepiped shape elongating in the longitudinal direction of the body unit 21 as a whole. The insertion unit 22 extends from one longitudinal-direction end of the body unit 21 in a direction substantially the same as the longitudinal direction of the body unit 21. The insertion unit 22 is a portion that is to be inserted into oral cavities in use of the biosensor 20.

The sensor unit 23 is attached near an area of the tip of the insertion unit 22. The sensor unit 23 is exposed on a surface, which faces downwards, among the outer surfaces of the insertion unit 22. In the first embodiment, the sensor unit 23 is a capacitive sensor which detects an electrostatic capacity value. The electrostatic capacity value is data for calculating the amount of water in an oral cavity. Specifically, the biosensor 20 converts an electrostatic capacity value to the amount of water in an oral cavity, and displays it on the display screen 21B. The amount of water is a type of biological information BI of a subject.

As illustrated in FIG. 2, the sensor unit 23 has a pair of electrodes 23A. The electrodes 23A have an interdigital shape. The pair of electrodes 23A have teeth disposed alternately. The pair of electrodes 23A functions as an electrode of a capacitor. In the sensor unit 23, the portion between the pair of electrodes 23A functions as a dielectric for the pair of electrodes 23A. The sensor unit 23 detects an electrostatic capacity value detected by the electrodes 23A due to the proximity or contact of a measurement target. In the case where the power supply of the biosensor 20 is ON, the sensor unit 23 detects an electrostatic capacity value even when a user is not performing a measurement operation.

A transparent film cover is attached to the sensor unit 23 in measurement. The film cover has such a size as to cover the entire sensor unit 23 and approximately the entire of the insertion unit 22. The transparent film cover is disposable. Specifically, a single film cover is used for a series of measurement for a single subject. In the first embodiment, it is assumed that a single film cover is used after the biosensor 20 is removed from the case 30 for measurement of the amount of water till the biosensor 20 is housed.

As illustrated in FIG. 1, the case 30 includes a case body 31, a conductor 32, and an input monitor 33.

The case body 31 has a box shape having a bottom base 31A, which is rectangular in plan view, and side walls 31B, which rise from the sides of the bottom base 31A. Therefore, corresponding to the elongating shape of the biosensor 20, the case body 31 has a shape extending longitudinally in one direction. In the description below, the direction, in which the bottom base 31A of the case body 31 is oriented, is assumed to be the top side of the case 30, and its opposite side is assumed to be the bottom side of the case 30.

As illustrated in FIG. 4, the height of the side walls 31B is substantially the same as the maximum dimension from the top surface 21A of the biosensor 20 to the bottom surface on the opposite side of the top surface 21A. The space between the side walls 31B in the longitudinal direction is larger than the maximum dimension in the longitudinal direction of the biosensor 20. The space between the side walls 31B in the lateral direction is larger than the maximum dimension in the lateral direction of the biosensor 20. That is, the space, which is defined by the side walls 31B and the bottom base 31A of the case body 31, may house the biosensor 20. The biosensor 20 is housed in the case 30 in such an orientation that the top surface 21A of the body unit 21 is opposite to the bottom base 31A of the case body 31.

As illustrated in FIG. 1, the conductor 32 has an approximately-rectangular planar shape. The material of the conductor 32 is an alloy having a main component of iron. “Having a main component of iron” means that the atomic percentage of iron in an alloy exceeds 50%. As illustrated in FIG. 4, the conductor 32 is disposed so as to be in contact with the sensor unit 23 when the biosensor 20 is housed in the case 30. Specifically, the conductor 32 is positioned in a portion of the surface of the bottom base 31A of the case body 31, which is on the boundary with a side wall 31B on one end side in the longitudinal direction. As illustrated in FIG. 1, the dimension of the conductor 32 in the lateral direction of the case 30 is substantially the same as that of the space between the side walls 31B in the lateral direction. That is, the conductor 32 extends over the short side of the case body 31 in the area defined by the side walls 31B and the bottom base 31A of the case body 31. As illustrated in FIG. 4, the thickness of the conductor 32 is equal to the length from the bottom base 31A of the case body 31 to the sensor unit 23 in the state in which the biosensor 20 is housed in the case 30.

The input monitor 33 is positioned on the surface, which faces upward, of a side wall 31B. Specifically, the input monitor 33 is positioned on the surface, which faces upward, of the side wall 31B that is positioned, in the longitudinal direction of the case body 31, on the side on which the conductor 32 is positioned. The input monitor 33 is a scanner which recognizes a barcode or a two-dimensional code.

### About the Electrical Configuration of the Biological Information Detection System

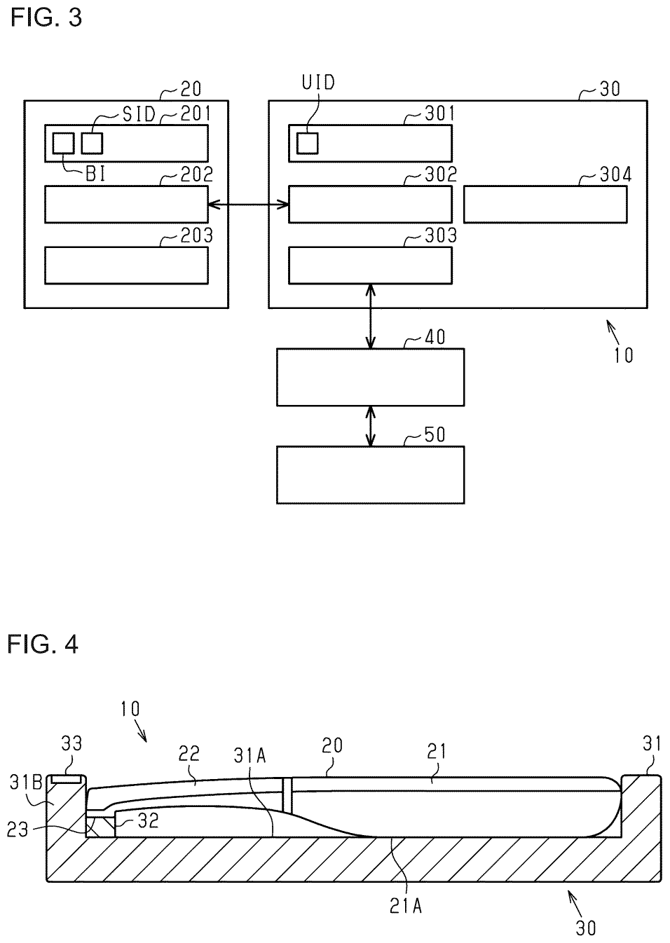

As illustrated in FIG. 3, the biosensor 20 includes a first storage unit 201, a first communication unit 202, and a sensor controller 203. The first storage unit 201, the first communication unit 202, and the sensor controller 203 are sometimes configured as a single chip or module, or are sometimes configured as individual chips or modules.

The first storage unit 201 is a storage medium readable by the sensor controller 203. The first storage unit 201 includes a read-only memory (ROM) which is only readable, a nonvolatile memory which is readable and writable, and a volatile memory which is readable and writable. The first storage unit 201 stores detected biological information BI. The first storage unit 201 stores sensor identification information SID specific to each biosensor 20. The sensor identification information SID is identified, for example, by using a combination of numbers, alphabets, and the like.

The first storage unit 201 stores, in the form of program data, various types of processing performed by the sensor controller 203. The first storage unit 201 stores, in advance, control values necessary for the various types of processing performed by the sensor controller 203.

The first communication unit 202 is capable of transmitting the biological information BI and the sensor identification information SID, which are stored in the first storage unit 201, to the case 30 through wireless communication. The first communication unit 202 is capable of transmitting information about the remaining amount of battery power of the biosensor 20, to the case 30 through wireless communication. The system of the wireless communication of the first communication unit 202 is, for example, Bluetooth®.

The sensor controller 203 is circuitry including one or more processors. The sensor controller 203 may be one or more dedicated hardware circuits, such as an application-specific integrated circuit (ASIC), or circuitry including a combination of these. The sensor controller 203 performs various types of processing, such as communication using the first communication unit 202 and reading and writing data from/to the first storage unit 201. The biosensor 20 also includes peripheral circuitry, such as a power supply circuit and a clock circuit (not illustrated).

In measurement of the amount of water, the sensor controller 203 converts an electrostatic capacity value, which has been detected by the sensor unit 23, to the amount of water by referring to a relational expression or the like stored in the first storage unit 201 in advance. The sensor controller 203 stores the calculated amount of water, as biological information BI detected by the biosensor 20, in the first storage unit 201.

The sensor controller 203 is capable of performing a housing determination process. The housing determination process is a process of repeatedly detecting whether the biosensor 20 has been housed in the case 30. That is, the sensor controller 203 repeatedly performs the housing determination process at predetermined intervals.

Specifically, in the housing determination process, when an electrostatic capacity value detected from the sensor unit 23 falls in a stipulated range, the sensor controller 203 determines that the biosensor 20 has been housed in the case 30. The stipulated range is predetermined as an electrostatic capacity value which may be detected when the sensor unit 23 is in contact with the conductor 32.

In the housing determination process, when the sensor controller 203 determines that the biosensor 20 has been housed in the case 30, the sensor controller 203 transmits, to the case 30 through the first communication unit 202, a housing signal indicating that the biosensor 20 has been housed in the case 30. Similarly, in the housing determination process, when the sensor controller 203 determines that the biosensor 20 has not been housed in the case 30, the sensor controller 203 transmits, to the case 30 through the first communication unit 202, a non-housing signal indicating that the biosensor 20 has not been housed in the case 30. As described above, the biosensor 20 is capable of communicating with the case 30 through wireless communication. Therefore, just after it is determined that the biosensor 20 has not been housed in the case 30, the first communication unit 202 may communicate with the case 30.

The sensor controller 203 is capable of performing a first counting process. The first counting process is a process of counting a first elapsed time from the time when the biosensor 20 is removed from the case 30. Specifically, when it is determined that the biosensor 20 has not been housed in the case 30 in a housing determination process, the sensor controller 203 starts counting the first elapsed time. In a housing determination process after the first counting process starts, when it is determined that the biosensor 20 has been housed in the case 30, the sensor controller 203 ends counting of the first elapsed time. That is, in the first counting process, the sensor controller 203 counts the first elapsed time from when the biosensor 20 is removed from the case 30, till the biosensor 20 is housed in the case 30. In restart of the first counting process, the first elapsed time is reset to zero.

When biological information BI is detected during execution of the first counting process, the sensor controller 203 stores, in the first storage unit 201, the detected biological information BI and the first elapsed time, which is obtained when the biological information BI is detected, in association with each other. In other words, the first storage unit 201 stores biological information BI and the first elapsed time, which is obtained when the biological information BI is detected, in association with other. When second biological information BI is detected during execution of the first counting process, the sensor controller 203 stores, in the first storage unit 201, the second biological information BI and the elapsed time, which is obtained when the second biological information BI is detected, in association with each other. The same is true for the case in which three or more pieces of biological information BI are detected during execution of the first counting process. “In association with each other” means that, between biological information BI and the first elapsed time, one of the pieces of data may be used to identify the other piece of data.

The case 30 includes a second storage unit 301, a second communication unit 302, a third communication unit 303, and a case controller 304.

The second storage unit 301 is a storage medium readable by the case controller 304. The second storage unit 301 includes a ROM which is only readable, a nonvolatile memory which is readable and writable, and a volatile memory which is readable and writable. The second storage unit 301 stores, in the form of program data, various types of processing performed by the case controller 304. The second storage unit 301 stores, in advance, control values necessary for the various types of processing performed by the case controller 304.

The second communication unit 302 receives biological information BI and sensor identification information SID which are transmitted by the first communication unit 202. The second communication unit 302 receives housing signals and non-housing signals transmitted by the first communication unit 202. The second communication unit 302 receives information about the remaining amount of battery power of the biosensor 20. These signals and pieces of information are stored in the second storage unit 301. The system of the wireless communication of the second communication unit 302 is the same as that of the first communication unit 202 of the biosensor 20.

The third communication unit 303 wirelessly transmits, to a server 40, information, such as the biological information BI and the sensor identification information SID which are received by the second communication unit 302. The server 40 is an exemplary external device. The third communication unit 303 allows communication in a range wider than the wireless communication range of the second communication unit 302. In other words, the communication range of the second communication unit 302 is narrower than the wireless communication range of the third communication unit 303. For example, the system of the wireless communication of the third communication unit 303 uses a cellular phone network. The server 40 has a central processing unit (CPU) and a ROM (not illustrated). The CPU of the server 40 executes programs stored in the ROM. Thus, the server 40 is capable of transmitting signals to a portable terminal 50 such as a smartphone owned by a user.

The case controller 304 is circuitry including one or more processors. The case controller 304 may be one or more dedicated hardware circuits such as an ASIC, or circuitry including a combination of these. The case controller 304 controls communication using the second communication unit 302 and communication using the third communication unit 303. The case controller 304 performs various types of processing such as reading and writing data from/to the second storage unit 301. The case 30 includes peripheral circuitry, such as a power supply circuit and a clock circuit (not illustrated).

The case controller 304 is capable of performing a determination process. The determination process is a process of repeatedly determining whether the biosensor 20 has been housed in the case 30. That is, the case controller 304 repeatedly performs the determination process at predetermined intervals. Specifically, in execution of a determination process, when the latest signal stored in the second storage unit 301 is a housing signal, the case controller 304 determines that the biosensor 20 has been housed in the case 30. Similarly, in execution of a determination process, when the latest signal, which is among received housing signals and non-housing signals and which is stored in the second storage unit 301, is a non-housing signal, the case controller 304 determines that the biosensor 20 has not been housed in the case 30. Thus, in the determination process, the case controller 304 repeatedly determines whether the biosensor 20 has been housed in the case 30 on the basis of the determination result in the housing determination process performed by the sensor controller 203 of the biosensor 20. Therefore, the case controller 304 determines whether the biosensor 20 has been housed in the case 30 on the basis of determination based on an electrostatic capacity value detected by the sensor unit 23 of the biosensor 20. In other words, in the determination process, the case controller 304 determines whether the biosensor 20 has been housed in the case 30 on the basis of an electrostatic capacity value detected by the sensor unit 23.

The case controller 304 is capable of performing a notification process. The notification process is performed when it is determined that the biosensor 20 has not been housed in the case 30 in the determination process. Specifically, the notification process is a process of outputting, to the server 40, the latest sensor identification information SID among the pieces of sensor identification information SID stored in the second storage unit 301, and information indicating that the biosensor 20 has not been housed in the case 30. When a notification process is performed, the server 40 notifies the portable terminal 50 that a specific biosensor 20 has not been housed in the case 30. The specific biosensor 20 is a sensor corresponding to the sensor identification information SID received in the notification process.

The case controller 304 is capable of performing a subject-information reception process. The subject-information reception process is a process of obtaining subject identification information UID specific to each subject. The subject identification information UID is comprised of a combination of numbers and alphabets which is assigned to each subject. When a subject-information reception process is performed, the input monitor 33 switches to the reading mode. In the reading mode, the input monitor 33 reads a barcode, a two-dimensional code, or the like assigned to each subject. Thus, the case controller 304 obtains subject identification information UID. The subject identification information UID is stored in the second storage unit 301.

The case controller 304 is capable of performing a second counting process. The second counting process is a process of counting a second elapsed time from a time when it is determined that the biosensor 20 has not been housed in the case 30 in the determination process. Specifically, when the latest signal, which is obtained when a determination process is performed, is switched to a non-housing signal, the case controller 304 starts counting the second elapsed time in the second counting process. After switching to a non-housing signal, when the latest signal, which is obtained when a determination process is performed, is switched to a housing signal, the case controller 304 ends counting of the second elapsed time. That is, in the second counting process, the case controller 304 counts the second elapsed time from when it is determined that the biosensor 20 has not been housed in the case 30 in the determination process, till, subsequently in the determination process, it is determined that the biosensor 20 has been housed in the case 30. In restart of the second counting process, the second elapsed time is reset to zero.

The case controller 304 performs an information reception process. The information reception process is a process of receiving biological information BI and sensor identification information SID by using the second communication unit 302 when it is detected that the biosensor 20 has been housed in the case 30 in the determination process. When the first counting process has been performed, the biological information BI is associated with the first elapsed time. Therefore, in the information reception process, the case controller 304 receives the first elapsed time, which is obtained when biological information BI is detected, in addition to the biological information BI and the sensor identification information SID. In the information reception process, information about the remaining amount of battery power of the biosensor 20 is received. The case controller 304 stores, in the second storage unit 301, the pieces of information received in the information reception process.

The case controller 304 is capable of performing an output process. The output process is a process of transmitting, from the third communication unit 303 to the server 40, the biological information BI, the sensor identification information SID, and the first elapsed time, which are received in the information reception process, in association with one another.

When a subject-information reception process has been performed, in the output process, the case controller 304 transmits, to the server 40, the subject identification information UID in addition to the information described above, in association with each other.

When the second counting process has been performed, in the output process, the case controller 304 transmits, to the server 40, the second elapsed time, which is obtained when it is determined that the biosensor 20 has been housed, in addition to the information described above, in association with each other.

In the output process, the case controller 304 transmits, to the server 40, the remaining amount of battery power of the biosensor 20 in addition to the information described above, in association with each other.

In the output process, the case controller 304 transmits, to the server 40, information indicating that the usage count of the biosensor 20 corresponding to the sensor identification information SID received in the information reception process is to be incremented by one, in addition to the information described above, in association with each other.

In the output process, the case controller 304 outputs, to the server 40, information, which indicates that the biosensor 20 has been housed in the case 30, in association with the sensor identification information SID received in the information reception process.

In response to reception of the first elapsed time, the server 40 calculates the time of detection of the biological information BI on the basis of the time of reception and the first elapsed time. The server 40 may transmit, to the portable terminal 50, the biological information BI and the time of detection of the biological information BI.

In response to reception of the second elapsed time, the server 40 calculates a sensor usage time, during which the biosensor 20 is out of the case 30, on the basis of the time of reception and the second elapsed time. The server 40 may transmit the sensor usage time to the portable terminal 50.

In response to reception of information indicating that the usage count of the biosensor 20 is to be incremented by one, the server 40 increments, by one, the accumulated usage count of the associated biosensor 20. The accumulated usage count corresponds to the number of covers consumed in measurement using the biosensor 20. The server 40 may transmit the accumulated usage count to the portable terminal 50.

The server 40 may transmit, to the portable terminal 50, information indicating that the biosensor 20 corresponding to the sensor identification information SID has been housed in the case 30, on the basis of the sensor identification information SID and the information indicating that the biosensor 20 has been housed in the case 30.

### About Operation of the First Embodiment

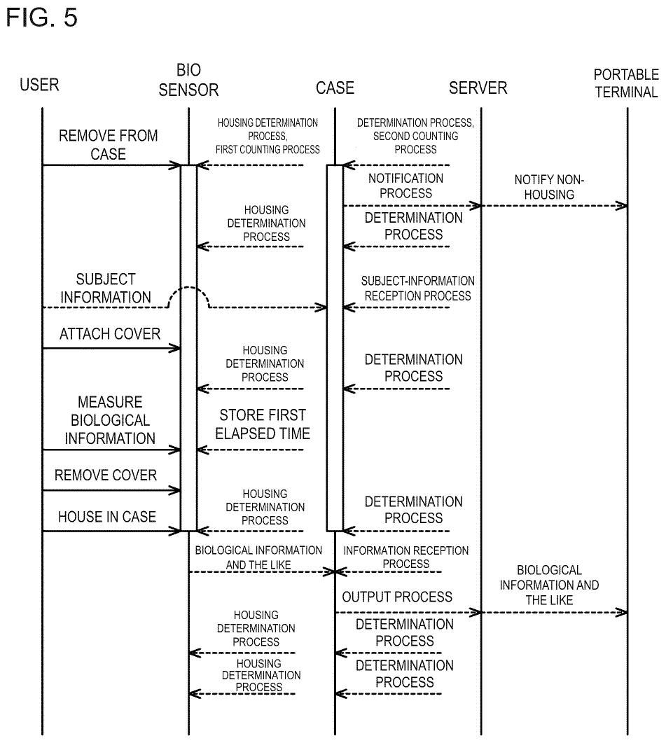

Referring to FIG. 5, an exemplary process performed by the biological information detection system 10 and the server 40 will be described. It is assumed that a user removes the biosensor 20 from the case 30 at a certain time point. At that time, in a housing determination process, the sensor controller 203 determines that the biosensor 20 has not been housed in the case 30. The first communication unit 202 of the biosensor 20 transmits a non-housing signal to the second communication unit 302 of the case 30. The sensor controller 203 performs a first counting process. The case controller 304, which has received the non-housing signal, determines that the biosensor 20 has not been housed in the case 30 in a determination process. The case controller 304 performs a second counting process. The case controller 304 repeatedly performs a determination process at predetermined intervals. In FIG. 5, some of the determination processes are not illustrated. The same is true for a housing determination process performed by the sensor controller 203.

Then, the case controller 304 performs a notification process. That is, the case controller 304 outputs, to the server 40, the latest sensor identification information SID among the pieces of sensor identification information SID stored in the second storage unit 301, and information indicating that the biosensor 20 has not been housed in the case 30.

In response to reception of the signal in the notification process, the server 40 notifies the portable terminal 50 that the specific biosensor 20 has not been housed in the case 30. For example, the portable terminal 50 displays, on its screen, information indicating that the specific biosensor 20 is being used.

After that, the case controller 304 performs a subject-information reception process. In execution of the subject-information reception process, the input monitor 33 switches to the reading mode. In switching to the reading mode, for example, the color of the input monitor 33 is changed. Thus, a user holds, over the input monitor 33, a barcode assigned to each subject. When the input monitor 33 detects the barcode successfully, the subject-information reception process ends.

After that, the user attaches a film cover on the sensor unit 23 of the biosensor 20. The user inserts the sensor unit 23 of the biosensor 20 into the oral cavity of a subject to measure, as biological information BI, the amount of water in the oral cavity of the subject.

In response to detection of biological information BI during execution of the first counting process, the sensor controller 203 stores, in the first storage unit 201, the first elapsed time and the biological information BI in association with other.

After that, the user removes the film cover from the biosensor 20. The user houses the biosensor 20 in the case 30.

At that time, in a housing determination process, the sensor controller 203 determines that the biosensor 20 has been housed in the case 30. The first communication unit 202 of the biosensor 20 transmits a housing signal to the second communication unit 302 of the case 30. After that, the sensor controller 203 ends the first counting process.

The case controller 304, which has received the housing signal, determines that the biosensor 20 has been housed in the case 30 in a determination process. The case controller 304 ends the second counting process, and stores the second elapsed time in the second storage unit 301.

The case controller 304 performs an information reception process. Specifically, the case controller 304 receives, from the biosensor 20, the biological information BI, the sensor identification information SID, the first elapsed time obtained when the biological information BI is detected, information about the remaining amount of battery power of the biosensor 20, and the like.

After that, the case controller 304 performs an output process. In the output process, in addition to the pieces of information received in the information reception process, the case controller 304 transmits, to the server 40, the subject identification information UID, the second elapsed time, information indicating that the usage count of the biosensor 20 is to be incremented by one, and information indicating that the biosensor 20 has been housed in the case 30, in association with one another.

After that, the server 40 calculates the time of detection of the biological information BI on the basis of the time of reception and the first elapsed time. The server 40 transmits, to the portable terminal 50, the biological information BI, the time of detection of the biological information BI, the sensor identification information SID, and the subject identification information UID in association with one another.

The server 40 calculates the sensor usage time, during which the biosensor 20 is out of the case 30, on the basis of the time of reception and the second elapsed time. The server 40 transmits, to the portable terminal 50, the sensor usage time, the sensor identification information SID, and the subject identification information UID in association with one another.

The server 40 increments, by one, the accumulated usage count of the biosensor 20 on the basis of the sensor identification information SID and the information indicating that the usage count of the biosensor 20 is to be incremented by one. The server 40 transmits, to the portable terminal 50, the accumulated usage count and the sensor identification information SID in association with each other.

The server 40 transmits, to the portable terminal 50, the sensor identification information SID and the remaining amount of battery power of the biosensor 20 in association with each other. The server 40 transmits, to the portable terminal 50, housing information indicating that the biosensor 20 has been housed in the case 30, and the sensor identification information SID in association with each other on the basis of the sensor identification information SID and the information indicating that the biosensor 20 has been housed in the case 30.

The portable terminal 50 displays, on its screen, the biological information BI and the time of detection of the biological information BI. The portable terminal 50 displays the sensor usage time on the screen. The portable terminal 50 displays the accumulated usage count on the screen. The portable terminal 50 displays the remaining amount of battery power of the biosensor 20 on the screen.

The portable terminal 50 displays, on the screen, information indicating that the specific biosensor 20 is not being used.

### About Effect of the First Embodiment

The first embodiment has effects described below.

(1-1) In the first embodiment, the server 40 receives the biological information BI and the sensor identification information SID. Thus, for example, information in the server 40 is confirmed through the portable terminal 50. Thus, a user of the portable terminal 50 may identify the biosensor 20 used in measurement of the biological information BI. Identification of the biosensor 20 enables management of information indicating that, for example, the received biological information BI is obtained in measurement on the specific subject associated with the biosensor 20.

(1-2) In the first embodiment, when the case controller 304 detects that the biosensor 20 has been housed in the case 30 in a determination process, the case controller 304 performs the information reception process. The information reception process is performed in the state in which the biosensor 20 has been housed in the case 30. Thus, this configuration prevents the connection between the first communication unit 202 and the second communication unit 302 from being interrupted in the information reception process.

(1-3) In the first embodiment, the case 30 includes the third communication unit 303. In contrast, the biosensor 20 does not include a long-distance wireless communication device for transmission to the server 40. Therefore, compared with a configuration, like the case 30, having a long-distance wireless communication device, the biosensor 20 may be designed with a reduction in size and weight.

(1-4) In the first embodiment, the case controller 304 performs the notification process. That is, the case 30 may transmit, to the server 40, information indicating that the biosensor 20 has been removed from the case 30. A user may confirm that the biosensor 20 has been removed from the case 30, recognizing that the biosensor 20 is being used.

(1-5) In the first embodiment, in the output process, the case controller 304 transmits, to the server 40, the subject identification information UID in addition to the biological information BI and the sensor identification information SID in association with one another. This configuration enables the server 40 to manage which biosensor 20 measures which subject's biological information BI, achieving more accurate management of biological information BI.

(1-6) In the first embodiment, in the information reception process, the case controller 304 receives, in addition to the biological information BI and the sensor identification information SID, the first elapsed time from activation of the first counting process till detection of the biological information BI. In the output process, the case controller 304 transmits, to the server 40, the first elapsed time in addition to the biological information BI and the sensor identification information SID, in association with one another. This configuration enables a user to confirm the time of detection of the biological information BI.

(1-7) In the first embodiment, in the output process, the case controller 304 transmits, to the server 40, the second elapsed time in addition to the biological information BI and the sensor identification information SID in association with one another. This configuration enables a user to confirm the sensor usage time from the time when the biosensor 20 is removed from the case 30 till the time when the biosensor 20 is housed in the case 30.

(1-8) In the first embodiment, the case controller 304 performs the determination process on the basis of an electrostatic capacity value detected by the sensor unit 23. This configuration enables the determination process to be performed by using the sensor unit 23 used for calculation of the biological information BI. Therefore, the biosensor 20 and the case 30 do not need a further mechanism for performing the determination process, achieving suppression of increase in size of the biosensor 20 and the case 30.

(1-9) In the first embodiment, in the output process, the case controller 304 transmits the remaining amount of battery power of the biosensor 20 to the server 40. Thus, a user confirms the remaining amount of battery power of the biosensor 20 which is used, facilitating taking measures such as charge of the biosensor 20.

(1-10) In the first embodiment, in the output process, the case controller 304 outputs information indicating that the usage count of the biosensor 20 is to be incremented by one. A user may confirm the accumulated usage count of the biosensor 20. The accumulated usage count corresponds to the number of consumed film covers. Therefore, the user may confirm the accumulated usage count to estimate the number of remaining film covers, allowing management of the count of film covers.

### Second Embodiment

A biological information detection system according to a second embodiment will be described below by referring to the drawings. Like the case of the first embodiment, in the drawings, components may be enlarged for illustration to facilitate understanding. The dimensional ratios of components may be different from actual ones or those in different drawings.

### About the Entire Configuration of the Biological Information Detection System

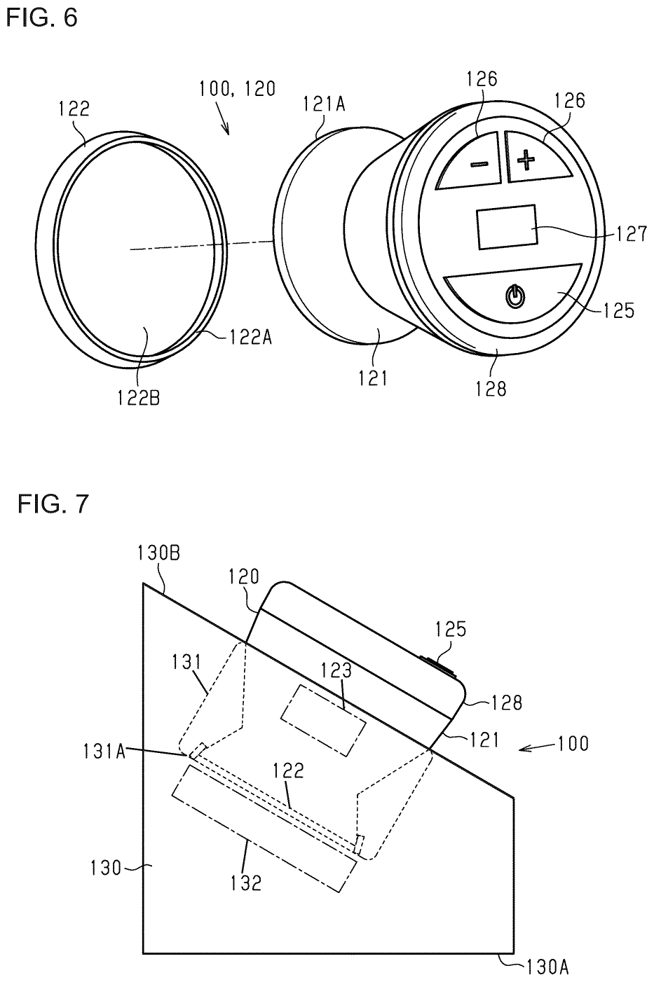

As illustrated in FIG. 6, a biological information detection system 100 includes a biosensor 120. In the second embodiment, the biosensor 120 is an electronic stethoscope which detects, as biological information BI of a subject, sound such as pulse sound emitted from a living body, and particularly detects pulse rates.

As illustrated in FIG. 7, the biosensor 120 includes a body unit 121, a sound collector 123, and a diaphragm 122.

As illustrated in FIG. 6, the body unit 121 has a cylindrical shape as a whole. Specifically, the outside diameter of a central portion in the central-axis direction of the body unit 121 is smaller than the outside diameter of each end portion in the central-axis direction of the body unit 121. In other words, the body unit 121 has a cylindrical shape in which the central portion is constricted. One end of the body unit 121 is closed. In contrast, the body unit 121 has an opening 121A on the other end. Thus, even with one end closed, a shape having an opening is expressed as a cylindrical shape. As illustrated in FIG. 7, the body unit 121 houses the sound collector 123. The sound collector 123 detects sound inside the body unit 121 for conversion into an electric signal.

As illustrated in FIG. 6, the diaphragm 122 is removable from the opening 121A of the body unit 121. “To be removable” means to be capable of being attached and removed without damage or irreversible deformation of the diaphragm 122 and the body unit 121. The diaphragm 122 has a frame 122A and a vibrating plate 122B. The frame 122A has a circular shape. The inside diameter of the frame 122A is substantially the same as the outside diameter on the other end of the body unit 121. Therefore, the frame 122A may be fit to the opening 121A of the body unit 121 from the outside in the radial direction. The vibrating plate 122B is a flexible film. The material of the vibrating plate 122B is, for example, synthetic resin or elastomer. The vibrating plate 122B is affixed to the frame 122A. Therefore, the vibrating plate 122B closes the opening of the frame 122A.

The biosensor 120 further includes a power supply button 125, two operation buttons 126, a display 127, and an indicator lamp 128.

The power supply button 125 is positioned on the end surface, which is opposite to the side on which the diaphragm 122 is attached, of the body unit 121. The power supply button 125 is exposed from the outer surface of the body unit 121. The power supply button 125 is a push down button, for example, for switching on/off the power supply of the biosensor 120.

The operation buttons 126 are positioned on the same end surface of the body unit 121 as that on which the power supply button 125 is exposed. The two operation buttons 126 are disposed adjacent to each other. The operation buttons 126 are exposed from the outer surface of the body unit 121. The operation buttons 126 are push down buttons for switching the setting or the like of the biosensor 120.

The display 127 is positioned on the same end surface of the body unit 121 as that on which the power supply button 125 is exposed. The display 127 is exposed from the outer surface of the body unit 121. The display 127 is capable of displaying various types of information. Specifically, the display 127 is capable of displaying at least three figures.

The indicator lamp 128 has a circular shape. The indicator lamp 128 is fit on the edge of the end surface of the body unit 121, which is opposite to the side on which the diaphragm 122 is attached. Light emitting diodes (LEDs) of red, green, and blue (not illustrated) are built in the indicator lamp 128. The LEDs of the colors are arranged along the circular shape of the indicator lamp 128. Therefore, the indicator lamp 128 may emit light of any color. The indicator lamp 128 may emit light in part.

As illustrated in FIG. 7, the biological information detection system 100 includes a case 130.

The case 130 has a square pillared shape as a whole. A lower surface 130A, which is one of the end surfaces of the case 130, is orthogonal to the central-axis line of the case 130. An upper surface 130B, which is the other end surface of the case 130, inclines at an angle of about 45° with respect to the central-axis line of the case 130. Therefore, when the case 130 is placed on a horizontal surface with the lower surface 130A down, the upper surface 130B faces upwards obliquely.

The case 130 has a housing space 131 for housing the biosensor 120. The housing space 131 forms a recess from the upper surface 130B in the direction orthogonal to the upper surface 130B. The housing space 131 has a cylindrical shape. The inside diameter of the housing space 131 is slightly larger than the outside diameter of the diaphragm 122 of the biosensor 120. The depth dimension of the housing space 131 is smaller than the dimension of the biosensor 120 in the direction of the central-axis line. When the biosensor 120 is inserted into the housing space 131 of the case 130 from the diaphragm 122 side, the diaphragm 122 faces a bottom surface 131A of the housing space 131. A portion of the biosensor 120 protrudes from the upper surface 130B of the case 130. Therefore, even in the state in which the biosensor 120 is housed in the housing space 131, the display 127 and the indicator lamp 128 of the biosensor 120 are exposed to the outside.

The case 130 has a sound emitter 132. The sound emitter 132 is positioned near the bottom surface 131A of the housing space 131 in the inside of the case 130. The sound emitter 132 is not exposed to the inside of the housing space 131. The sound emitter 132 emits predetermined sounds into the housing space 131. Each sound emitted by the sound emitter 132 has, for example, a frequency outside the typical range audible by persons, that is, 20 Hz or less, or 20 kHz or greater. The sound emitter 132 emits each sound intermittently. Specifically, the sound emitter 132 alternately repeats the state in which each sound is emitted and the state in which each sound is not emitted. The repetition period at that time is, for example, in the order of several tens of milliseconds to several hundreds of milliseconds. The magnitude of each sound emitted by the sound emitter 132 is extremely small.

### About the Electrical Configuration of the Biological Information Detection System

The biosensor 120 includes the first storage unit 201, the first communication unit 202, and the sensor controller 203. The basic configuration of the first storage unit 201, the first communication unit 202, and the sensor controller 203 is substantially the same as that in the case of the first embodiment illustrated in FIG. 3. Therefore, the description about points of the first storage unit 201, the first communication unit 202, and the sensor controller 203, which are common to those in the first embodiment, will be skipped or simplified below.

The first storage unit 201 stores detected biological information BI. The first storage unit 201 stores sensor identification information SID specific to each biosensor 20. The first communication unit 202 is capable of transmitting, to the case 30 through wireless communication, the biological information BI and the sensor identification information SID which are stored in the first storage unit 201.

The sensor controller 203 calculates a pulse rate on the basis of an electric signal obtained through conversion performed by the sound collector 123. Specifically, the sensor controller 203 determines peaks of the magnitude of the sound, which is emitted by a living body, on the basis of change, in a certain period, of the sound. The sensor controller 203 detects a per-minute pulse rate, for example, on the basis of the intervals of the peaks of the magnitude of the sound. The sensor controller 203 stores, in the first storage unit 201, the calculated pulse rate as biological information BI detected by the biosensor 20.

The sensor controller 203 controls the color of light, which is emitted by the indicator lamp 128, in accordance with the pulse rate as biological information BI. For example, when the pulse rate is 200 or greater, the sensor controller 203 sets the color of the light, which is emitted by the indicator lamp 128, to red. When the pulse rate is less than 200 or 100 or greater, the sensor controller 203 sets the color of the light, which is emitted by the indicator lamp 128, to green. When the pulse rate is less than 100, the sensor controller 203 sets the color of the light, which is emitted by the indicator lamp 128, to blue.

The sensor controller 203 is capable of performing a housing determination process and the first counting process. The first counting process and processes accompanied by this, which are substantially the same as those in the first embodiment, will not be described.

The housing determination process is a process of repeatedly detecting whether the biosensor 120 has been housed in the case 130. That is, the sensor controller 203 repeatedly performs the housing determination process at predetermined intervals. Specifically, in the housing determination process, the sensor controller 203 determines whether a predetermined first stipulated sound has been detected repeatedly through the sound collector 123. Assume that a normal diaphragm 122 is attached to the body unit 121 of the biosensor 120 and that the biosensor 120 is normally housed in the housing space 131 of the case 130. The first stipulated sound is determined, for example, on the basis of a test or a simulation, as a sound of the sound emitter 132 which is detectable by the sound collector 123 under the condition described above. Therefore, the first stipulated sound is determined on the basis of a sound emitted by the sound emitter 132. When the first stipulated sound is detected continuously a stipulated number of times or more, the sensor controller 203 determines that the biosensor 120 has been housed in the case 130. The stipulated number of times may be determined, for example, as several times to several tens of times.

In the housing determination process, the sensor controller 203 determines whether a predetermined second stipulated sound has been detected repeatedly through the sound collector 123. Assume that the diaphragm 122 has not been attached to the body unit 121 of the biosensor 120 and that the biosensor 120 has been housed in the housing space 131 of the case 130. The second stipulated sound is determined, for example, on the basis of a test or a simulation, as a sound of the sound emitter 132 which is detectable by the sound collector 123 under the condition described above. Thus, although the same sound emitter 132 is used as a sound source, the first stipulated sound and the second stipulated sound are different in that the sound is obtained through the diaphragm 122 or not. When the second stipulated sound is detected continuously the stipulated number of times or more, the sensor controller 203 determines that the biosensor 120 has been housed in the case 130. The stipulated number of times at that time is the same count as that in the case of the first stipulated sound.

In contrast, when neither the first stipulated sound nor the second stipulated sound is detected continuously the stipulated number of times or more, the sensor controller 203 determines that the biosensor 120 has not been housed in the case 130.

In the housing determination process, when the sensor controller 203 determines that the biosensor 120 has been housed in the case 130 on the basis of repeated detection of the first stipulated sound, the sensor controller 203 transmits a first signal to the case 130 through the first communication unit 202. Similarly, in the housing determination process, when the sensor controller 203 determines that the biosensor 120 has been housed in the case 130 on the basis of repeated detection of the second stipulated sound, the sensor controller 203 transmits a second signal to the case 130 through the first communication unit 202. Therefore, each of the first signal and the second signal is a type of housing signal. When the second signal is outputted, the sensor controller 203 emits light of the indicator lamp 128 for a predetermined period. For example, the sensor controller 203 blinks the indicator lamp 128 in red over several seconds.

In contrast, in the housing determination process, when the sensor controller 203 determines that the biosensor 20 has not been housed in the case 130, the sensor controller 203 transmits, to the case 130 through the first communication unit 202, a non-housing signal indicating that the biosensor 20 has not been housed in the case 130.

The case 130 includes the second storage unit 301, the second communication unit 302, the third communication unit 303, and the case controller 304. The basic configuration of the second storage unit 301, the second communication unit 302, the third communication unit 303, and the case controller 304 is substantially the same as that in the first embodiment in FIG. 3. Therefore, the description about the points of the second storage unit 301, the second communication unit 302, the third communication unit 303, and the case controller 304, which are common to those in the first embodiment, will be skipped or simplified below.

The second communication unit 302 receives biological information BI and sensor identification information SID which are transmitted by the first communication unit 202. The second communication unit 302 receives first signals, second signals, and non-housing signals transmitted by the first communication unit 202. These signals are stored in the second storage unit 301. The third communication unit 303 wirelessly transmits, to the server 40 which is an external device, information, such as biological information BI and sensor identification information SID, which is received by the second communication unit 302.

The case controller 304 is capable of performing a determination process. The determination process is a process of repeatedly determining whether the biosensor 120 has been housed in the case 130. That is, the case controller 304 repeatedly performs the determination process at predetermined intervals. Specifically, when, in execution of a determination process, the latest signal stored in the second storage unit 301 is a first signal, the case controller 304 determines that the biosensor 120 has been housed in the case 130. That is, when a first signal has been received, the case controller 304 determines that the biosensor 120 has been housed in the case 130.

When, in execution of a determination process, the latest signal stored in the second storage unit 301 is a second signal, the case controller 304 determines that, although the biosensor 120 has been housed in the case 130, there is an abnormality in the diaphragm 122. That is, when a second signal has been received, the case controller 304 determines that there is an abnormality in the diaphragm 122. “Abnormality” described here is an expediential expression for describing the state in contrast with the normal state in which the diaphragm 122 is attached to the body unit 121. Therefore, use of the biosensor 120 in an “abnormal” state is not excluded.

In contrast, when, in execution of a determination process, the latest signal stored in the second storage unit 301 is a non-housing signal, the case controller 304 determines that the biosensor 120 has not been housed in the case 130.

Thus, in the determination process, the case controller 304 repeatedly determines whether the biosensor 20 has been housed in the case 30 on the basis of the determination result in a housing determination process performed by the sensor controller 203 of the biosensor 20.

The case controller 304 is capable of performing the notification process, the subject-information reception process, the second counting process, and the information reception process. These processes and processes accompanied by these, which are substantially the same as those in the first embodiment, will not be described.

The case controller 304 is capable of performing an output process. The output process is a process of transmitting, from the third communication unit 303 to the server 40, the biological information BI, the sensor identification information SID, the first elapsed time, the subject identification information UID, the second elapsed time, and the like, which are received in the information reception process, in association with one another.

When the case controller 304 determines that the biosensor 120 has been housed in the case 130 on the basis of storage of a first signal, the case controller 304 transmits the biological information BI, the sensor identification information SID, and the like from the third communication unit 303 to the server 40 in the output process. In contrast, when the case controller 304 determines that the biosensor 120 has been housed in the case 130 on the basis of storage of a second signal, the case controller 304 transmits, from the third communication unit 303 to the server 40, information indicating that there in an abnormality in the diaphragm 122, in addition to the biological information BI, the sensor identification information SID, and the like in association with one another in the output process. In other words, when it is determined that there is an abnormality in the diaphragm 122 in the determination process, the case controller 304 transmits, from the third communication unit 303 to the server 40, information indicating the determination result. The server 40, which has received the information, displays an attention attracting message, such as “The detection result from the biosensor of No. X is obtained without a diaphragm,” or “No diaphragm is attached to the biosensor of No. X,” for example, on a display connected to the server 40.

### About Operation of the Second Embodiment

An exemplary process performed by the biological information detection system 100 and the server 40 will be described. Description about points common to those in the first embodiment will be skipped or simplified.

Assume that, at a certain time point, a user removes a biosensor 120 from the case 130. The user removes the old diaphragm 122 from the body unit 121 of the biosensor 120, and attaches a new diaphragm 122. After that, the user measures the pulse rate of a subject by using the biosensor 120, and houses the biosensor 120 in the case 130.

At that time, the diaphragm 122 is attached correctly. Thus, the sound collector 123 repeatedly detects the first stipulated sound. Therefore, the sensor controller 203 determines that the biosensor 20 has been housed in the case 30, and outputs a first signal. The case controller 304, which has received the first signal, transmits, to the server 40, the biological information BI and the like which are received in the information reception process.

In contrast, assume that, after the user removes the old diaphragm 122 from the body unit 121 of the biosensor 120, the user forgets to attach a new diaphragm 122. In this case, the user measures the pulse rate of a subject by using the biosensor 120 to which a diaphragm 122 has not been attached, and houses the biosensor 120, as it is, in the case 130.

At that time, since no diaphragm 122 is attached, the sound collector 123 repeatedly detects the second stipulated sound. Therefore, the sensor controller 203 determines that there is an abnormality in the diaphragm 122 although the biosensor 20 has been housed in the case 30, and outputs a second signal. The case controller 304, which has received the second signal, transmits, to the server 40, information indicating that there is an abnormality in the diaphragm 122, in addition to the biological information BI and the like which are received in the information reception process.

### About Effect of the Second Embodiment

The second embodiment has effects described below, in addition to the effects of (1-1) to (1-7) and (1-9) of the first embodiment.

(2-1) In the second embodiment, when the sound collector 123 of the biosensor 120 detects the first stipulated sound, the sensor controller 203 outputs a first signal. When a first signal has been received, the case controller 304 determines that the biosensor 120 has been housed in the case 130. The sound collector 123, which is necessary as an electronic stethoscope, is used to determine whether the biosensor 120 has been housed in the case 130. Thus, no other additional detection devices are necessary for the determination.

(2-2) In the second embodiment, when the sound collector 123 of the biosensor 120 detects the second stipulated sound, the sensor controller 203 outputs a second signal. When a second signal has been received, the case controller 304 determines that there is an abnormality in the diaphragm 122. Therefore, the biosensor 120 is prevented from continuing to be used with occurrence of an abnormality such as the state in which no diaphragm 122 is attached.

(2-3) In the second embodiment, when the case controller 304 determines that there is an abnormality in the diaphragm 122, the case controller 304 transmits, to the server 40, information indicating this. Therefore, for example, when there are multiple biosensors 120, attachment and detachment of the diaphragms 122 of the biosensors 120 may be collectively managed on the basis of the information stored in the server 40.

(2-4) In the second embodiment, when the sound collector 123 of the biosensor 120 detects the second stipulated sound, the sensor controller 203 emits light of the indicator lamp 128 for a predetermined period. According to this, without referring to information in the server 40, a user may confirm whether a diaphragm 122 has been attached to the biosensor 120 when the user houses the biosensor 120 in the case 130.

### Modified Examples

The embodiments and modified examples described below may be carried out in combination with each other in the scope having no technical contradiction. A modified example of the biosensor 20 and the case 30 in the first embodiment may be applied to the biosensor 120 and the case 130 in the second embodiment as long as there is no technical contradiction. Similarly, a modified example of the biosensor 120 and the case 130 in the second embodiment may be applied to the biosensor 20 and the case 30 in the first embodiment as long as there is no technical contradiction.

The shape of the biosensor according to each embodiment may be changed. The shape may be set appropriately in accordance with the type of biological information BI that is to be detected, assumed usage, and the like

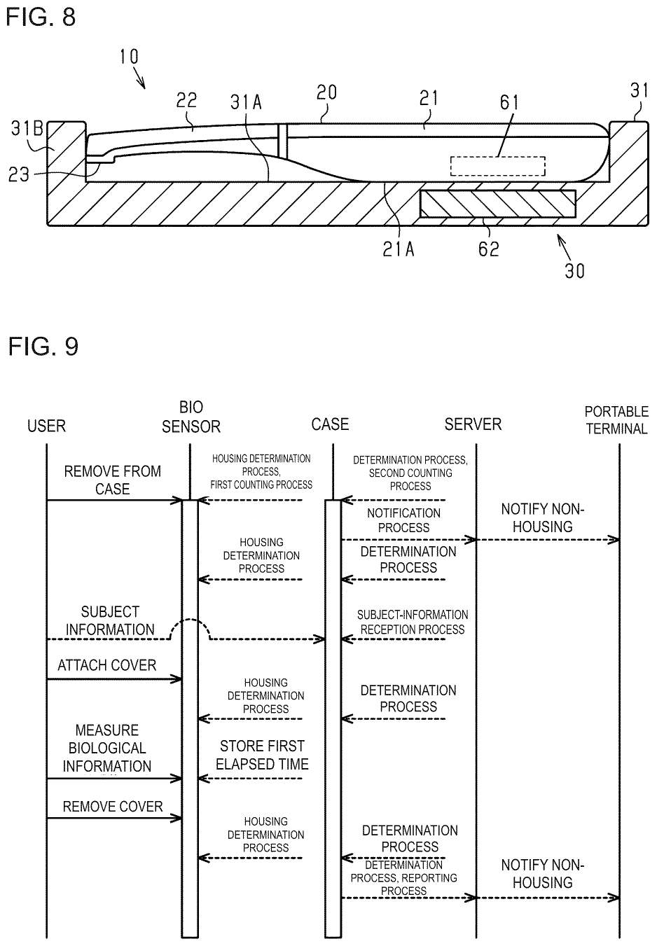

In the first embodiment, the case 30 does not necessarily have the conductor 32. In this case, any configuration may be employed as long as, in the determination process, the case controller 304 is capable of determining whether the biosensor 20 has been housed in the case 30 by using a method without use of the conductor 32. For example, the case 30 may have a pressure sensor. On the basis of application of pressure to the pressure sensor when the biosensor 20 has been housed in the case 30, the case controller 304 may perform the determination process. In addition, for example, in the example illustrated in FIG. 8, the biosensor 20 includes an NFC transmitter 61. NFC is an abbreviation of Near Field Communication. The NFC transmitter 61 is built in the body unit 21 of the biosensor 20. In this example, the case 30 includes an NFC receiver 62. The NFC receiver 62 is built in the case 30 at a position where the NFC receiver 62 faces the NFC transmitter 61 when the biosensor 20 is housed in the case 30. When the NFC transmitter 61 comes closer, the NFC receiver 62 is capable of receiving a signal from the NFC transmitter 61. Therefore, the NFC receiver 62 receives, from the NFC transmitter 61, a signal indicating that the biosensor 20 has been housed in the case 30. Thus, the case controller 304 may determine that the biosensor 20 has been housed in the case 30 in the determination process. In contrast, the NFC receiver 62 does not receive, from the NFC transmitter 61, a signal indicating that the biosensor 20 has been housed in the case 30. Thus, the case controller 304 may determine that the biosensor 20 has not been housed in the case 30 in the determination process. When the NFC transmitter 61 comes close to a certain degree, the NFC receiver 62 is capable of receiving information from the NFC transmitter 61. Therefore, in housing the biosensor 20 in the case 30, strict positioning of these is not necessary.

In this example, the NFC transmitter 61 may have the functions as the first communication unit 202. In this case, the biosensor 20 does not necessarily include the first communication unit 202. Similarly, in this example, the NFC receiver 62 may have the functions as the second communication unit 302. In this case, the case 30 does not necessarily include the second communication unit 302.

In the example in FIG. 8, the NFC receiver 62 is desirably connected to a power source. That is, the NFC receiver 62 desirably has a feeding function. If the NFC receiver 62 has a feeding function, the biosensor 20 may be charged in the state in which the biosensor 20 is housed in the case 30.

In the first embodiment, the case controller 304 may be capable of performing a reporting process of outputting information indicating that the biosensor 20 has not been housed in the case 30. For example, in the example in FIG. 9, like the example in FIG. 5, assume that a user measures biological information BI by using the biosensor 20, and removes the film cover from the biosensor 20. In the example in FIG. 9, after that, assume that the user forgets to house the biosensor 20 in the case 30. In this case, when the second elapsed time, which is being counted in the second counting process, exceeds a predetermined stipulated period, the case controller 304 performs the reporting process of outputting information indicating that the biosensor 20 has not been housed in the case 30. The stipulated period is, for example, from several minutes to several tens of minutes. In the example in FIG. 9, in the reporting process, the case controller 304 outputs, to the server 40, information indicating that the biosensor 20 has not been housed in the case 30. The server 40, which has received the information, reports, to the portable terminal 50, that the biosensor 20 has not been housed in the case 30. According to this configuration, when the biosensor 20 has not been housed in the case 30 for the stipulated period or longer, the user may detect this state. According to this, occurrence of the state in which, for example, the biosensor 20 is not housed in the case 30 and is lost may be suppressed.

In the example in FIG. 9, the output destination of the information indicating that the biosensor 20 has not been housed in the case 30 is not limited to the server 40. For example, if the case 30 includes a reporting device such as a speaker, the case controller 304 may output the information to the reporting device. In this case, the reporting device, which outputs beeper sound, enables a user to be notified that the biosensor 20 has not been housed in the case 30 for the stipulated period. Further, the information indicating that the biosensor 20 has not been housed in the case 30 may be directly transmitted to the portable terminal 50, not through the server 40.

The case controller 304 may perform the information reception process regardless of the result of a determination process. For example, in the case where the communication system of the second communication unit 302 is Bluetooth®, when the second communication unit 302 and the first communication unit 202 are in the connection state, the case controller 304 may perform the reception information process.

The case controller 304 does not necessarily transmit the second elapsed time in the output process. That is, the case controller 304 may skip the second counting process. The second counting process may be performed by the sensor controller 203. In this case, in the information reception process, the case controller 304 may obtain the second elapsed time from the biosensor 20.

The case controller 304 may skip the notification process. In other words, when the biosensor 20 has not been housed in the case 30, the case controller 304 does not necessarily transmit, to the server 40, information indicating that the biosensor 20 has been housed in the case 30.

In the information reception process, the case controller 304 does not necessarily receive the first elapsed time which is a time until detection of biological information BI. That is, the sensor controller 203 may skip the first counting process.

The first storage unit 201 in the biosensor 20 may be configured with multiple storage media. That is, in the biosensor 20, a storage medium storing biological information BI may be different from one storing sensor identification information SID.

When biological information BI is not measured appropriately from a subject, the biosensor 20 may obtain, as biological information BI, measurement error information instead of the amount of water. In this case, the case controller 304 may receive measurement error information in the information reception process. In this case, in the output process, the case controller 304 may transmit, from the third communication unit 303 to the server 40, biological information BI and sensor identification information SID, which are measurement error information received in the information reception process, in association with each other. A user may realize that the measurement has not been performed appropriately by checking the measurement error information.

The case controller 304 may skip the subject-information reception process. The case controller 304 may receive subject identification information UID from the biosensor 20 to perform the subject-information reception process. In this case, the biosensor 20 preferably may include an input device. A user inputs subject identification information UID through the input device. Thus, subject identification information UID may be inputted to the biosensor 20. When the subject-information reception process is skipped and, for example, when subject identification information UID is inputted to the biosensor 20 directly, the case 30 does not necessarily include the input monitor 33.

The time when the subject-information reception process is performed is not limited to one in the example according to the embodiments. For example, the subject-information reception process may be performed after measurement of biological information BI, or may be performed before the biosensor 20 is removed from the case 30.

The biosensor 20 is not limited to a water-amount measurement device as in the first embodiment, or an electronic stethoscope as in the second embodiment. Any configuration may be employed as long as the biosensor 20 is a device which is capable of detecting biological information BI of a subject. For example, the biosensor 20 may be a thermometer or the like which detects a body temperature as biological information BI. When the biosensor 20 is of a type in which a capacitive sensor is not used in measurement of biological information BI, the biosensor 20 or the case 30 may further include a capacitive sensor so that a determination process using the capacitive sensor is performed. In addition, in a method without use of a capacitive sensor, the determination process may be performed. For example, when the biosensor 20 is a thermometer, the biosensor 20, which has detected a temperature outside a predetermined stipulated range, may determine that it has been housed in the case 30. The stipulated range is determined, for example, as a value of 34 degrees or higher and less than 43 degrees, which may be obtained as the body temperature of a living body. For example, the case 30 may include a heater which keeps a temperature outside the stipulated range, and the biosensor 20, which has detected the temperature of the heater, may detect that it is housed in the case 30.

For example, assume that the biosensor 20 is a pulsimeter which is capable of detecting an electric pulse signal. In this case, the biosensor 20, which has detected an electric pulse signal outside a predetermined stipulated range, may determine that it has been housed in the case 30. The stipulated range is determined, for example, as an electric pulse signal which is possibly a pulse of a living body. For example, the case 30 may include a source which produces a pulse signal outside the stipulated range, and the biosensor 20, which has detected the pulse signal, may determine that it has been housed in the case 30.

The external device is not limited to the server 40. The external device may be, for example, a personal computer or the portable terminal 50.

The configuration of the biosensor 20 is not limited to the example in the embodiment. For example, the biosensor 20 may exclude the display screen 21B. The biological information BI may be displayed only through the portable terminal 50.

In the first embodiment, the material of the conductor 32 is not limited to an alloy having a main component of iron. Desirably, the material of the conductor 32 may be such a material that the electrostatic capacity value detected when the sensor unit 23 is in contact with the conductor 32 is far from the electrostatic capacity value detected when biological information BI is measured.

In the first embodiment, the shape of the case 30 is not limited to the example in the embodiment. Any shape may be employed as long as the case 30 may house the biosensor 20. For example, the case 30 may have such a shape that the biosensor 20 may be housed so that a part, including the sensor unit 23 and the insertion unit 22, is covered.

The wireless system of the first communication unit 202 and the second communication unit 302 is not limited to the example of the embodiments. The first communication unit 202 and the second communication unit 302 may perform wired communication.

In the information reception process, the case controller 304 does not necessarily receive the remaining amount of battery power of the biosensor 20.

In the output process, the case controller 304 does not necessarily transmit, to the server 40, information indicating that the usage count of the biosensor 20 corresponding to the sensor identification information SID is to be incremented by one.

In the second embodiment, the manner of emitting light of the indicator lamp 128 may be changed appropriately. For example, the light may blink in accordance with the pulse of a subject which has been detected by the biosensor 120. In addition, for example, a specific light may be emitted when the power supply of the biosensor 120 is turned on and detection of biological information BI is ready.

In the second embodiment, when the case controller 304 determines that the biosensor 120 has been housed in the case 130 on the basis of storage of the second signal, the case controller 304 does not necessarily output biological information BI to the server 40. If use of the biosensor 120 with the diaphragm 122 unattached is not assumed, the reliability of biological information BI in such a state may fail to be ensured. In this case, in view of management of subjects, it may be preferable that biological information BI is not transmitted to the server 40.

In the second embodiment, one or more of the components selected from the power supply button 125, the two operation buttons 126, the display 127, and the indicator lamp 128 of the biosensor 120 may be excluded. For example, the display 127 and the indicator lamp 128 may be excluded from the biosensor 120, and biological information BI may be confirmed only through the server 40.

In the second embodiment, the sound emitter 132 of the case 130 may be positioned anywhere. The position of the sound emitter 132 may be any as long as the sound emitter 132 may emit sound to the housing space 131 of the case 130.

In the second embodiment, the sound emitted by the sound emitter 132 is not limited to the example described above. For example, the sound emitted by the sound emitter 132 may be audible sound or may have a frequency changing on a case-by-case basis. However, the sound emitted by the sound emitter 132 may be a sound which does not occur as environmental sound in an environment in which the biosensor 120 is used, or a sound which does not occur as pulse sound of subjects which is detected by the biosensor 120.

In the second embodiment, the second stipulated sound is determined as a sound of the sound emitter 132 which is detected by the sound collector 123 in the state in which the diaphragm 122 is not attached to the body unit 121 of the biosensor 120. The configuration is not limited to this. For example, the second stipulated sound may be determined as a sound of the sound emitter 132 which is detected by the sound collector 123 in the state in which the diaphragm 122 is torn or is dirty excessively.

The second stipulated sound is not necessarily determined as a sound based on the sound emitted by the sound emitter 132. For example, the second stipulated sound may be determined as a sound having less than a predetermined magnitude, or, on the contrary, may be determined as a sound having greater than the predetermined magnitude.

In the second embodiment, the sensor controller 203 does not necessarily emit light of the indicator lamp 128 when the sound collector 123 of the biosensor 120 detects the second stipulated sound.

In the second embodiment, the case controller 304 does not necessarily output information to the server 40 when it is determined that there is an abnormality in the diaphragm 122. Also in this case, if light is emitted from the indicator lamp 128 as in the second embodiment, a user may realize an abnormality in the diaphragm 122.

In the second embodiment, in some cases, for example, the states, that is, the normal state in which the diaphragm 122 has been attached to the body unit 121 and the abnormal state in which the diaphragm 122 has not been attached to the body unit 121, are intentionally used differently in accordance with the position where sound emitted by a living body is detected. In this case, when the case controller 304 in the second embodiment determines that the biosensor 120 has been housed in the case 130 on the basis of storage of the first signal, the case controller 304 may transmit, to the server 40, information indicating that the diaphragm 122 has been attached. In the case of this modified example, it is preferable that the server 40 stores biological information BI in such a manner that whether the biological information BI is detected with or without the diaphragm 122 attached is recognized.

In the second embodiment, the process about detection of the second stipulated sound which is performed by the sensor controller 203 may be skipped. Even in this case, biological information BI obtained with the diaphragm 122 attached is transmitted to the case 130.

In the second embodiment, the biological information BI is not limited to a pulse rate. For example, recorded data of a sound detected by the sound collector 123 of the biosensor 120 may be used as biological information BI.

In a test device as described in Japanese Unexamined Patent Application Publication No. 2010-279701, when biological information is transmitted to the external device before the biosensor is housed in the case, it is difficult to determine whether the biosensor is being used or the biosensor housed in the case is not being used. In Japanese Unexamined Patent Application Publication No. 2010-279701, the timing when biological information, which has been obtained by the sensor obtaining biological information, is outputted to the external device is not discussed. In the first embodiment, when, in the determination process, the case controller 304 detects that the biosensor 20 has been housed in the case 30, the case controller 304 receives biological information BI through the second communication unit 302, and transmits the biological information BI from the third communication unit 303 to the server 40. This configuration enables the case 30 to output biological information BI to the server 40 in response to the state in which the biosensor 20 has been housed. That is, through detection of output of biological information BI to the external device, the state in which the biosensor 20 has been housed in the case 30 may be confirmed. Thus, the case controller 304's process of receiving sensor identification information SID from the biosensor 20 is not necessary in view of outputting biological information BI to the external device in response to the state in which the biosensor 20 has been housed in the case 30. In addition, the case controller 304's process of transmitting, to the server 40 which is an external device, biological information BI and sensor identification information SID in association with each other is also not necessary. The device, which outputs biological information BI to an external device, is not limited to the case 30 in view of outputting biological information BI to the external device in response to the state in which the biosensor 20 has been housed in the case 30. For example, when the biosensor 20 has been housed in the case 30, the biosensor 20 may output biological information BI to the external device. In this case, the biosensor 20 desirably includes a communication unit corresponding to the third communication unit 303. For these points, the same is true for the second embodiment.

### APPENDIX

The technical idea, which may be derived from the embodiments and the modified examples, will be described below.

A biological information detection system includes a biosensor that detects biological information of a subject, and a case that is capable of housing the biosensor. The biosensor includes a first storage unit that stores the detected biological information, and a first communication unit that transmits the biological information stored in the first storage unit. The case includes a second communication unit that receives the biological information transmitted by the first communication unit, a third communication unit that wirelessly transmits, to an external device, the biological information received by the second communication unit, and a case controller that controls communication through the second communication unit and communication through the third communication unit. The case controller is capable of performing a determination process of detecting whether the biosensor has been housed in the case, an information reception process of receiving the biological information by using the second communication unit when, in the determination process, the state in which the biosensor has been housed in the case is detected, and an output process of transmitting, from the third communication unit to the external device, the biological information received in the information reception process. The second communication unit has a communication range narrower than the wireless communication range of the third communication unit.

A biological information detection system includes a biosensor that detects biological information of a subject, and a case that is capable of housing the biosensor. The biosensor includes a first storage unit that stores the detected biological information, an externally-communicating unit that wirelessly transmits, to an external device, the biological information stored in the first storage unit, and a sensor controller that is capable of performing a housing determination process of determining whether the biosensor has been housed in the case. The sensor controller is capable of performing an output process of transmitting the biological information from the externally-communicating unit to the external device when, in the housing determination process, the state in which the biosensor has been housed in the case is detected. cm What is claimed is:

1. A biological information detection system comprising:
a biosensor configured to detect biological information of a subject; and
a case configured to house the biosensor,
wherein the biosensor comprises:
a first storage configured to store the detected biological information and sensor identification information specific to the biosensor, and
a first transmitter configured to transmit the biological information and the sensor identification information which are stored in the first storage, and
wherein the case comprises:
a receiver configured to receive the biological information and the sensor identification information which are transmitted by the transmitter,
a second transmitter configured to wirelessly transmit, to an external device, the biological information and the sensor identification information which are received by the receiver, in association with each other, and
a case controller configured to control communication through the receiver and communication through the second transmitter.

2. The biological information detection system according to claim 1, wherein the case controller is further configured to:
detect whether the biosensor is housed in the case, and
control communication through the receiver in response to detecting that the biosensor has been housed in the case.

3. The biological information detection system according to claim 2, wherein the case controller:
comprises a second storage configured to store the sensor identification information received by the receiver, and
is further configured to output, to the external device, latest sensor identification information and information indicating that the biosensor has not been housed in the case, in response to detecting that the biosensor has not been housed in the case, the latest sensor identification information being sensor identification information stored in the second storage.

4. The biological information detection system according to claim 1,
wherein the case controller is further configured to control communication through the receiver to obtain subject identification information specific to the subject, and
wherein the case controller is configured to, as part of the outputting, control communication through the second transmitter to transmit, to the external device, the subject identification information in addition to the biological information and the sensor identification information in association with one another.

5. The biological information detection system according to claim 1,
wherein the biosensor further comprises a sensor controller configured to determine whether the biosensor has been housed in the case,
wherein the sensor controller is configured to count a first elapsed time from when the biosensor is removed from the case, until the biosensor is housed in the case,
wherein, in response to detection of the biological information while counting the first elapsed time, the first storage is configured to store the detected biological information and the first elapsed time obtained when the biological information is detected, in association with each other, and
wherein, the case controller is configured to, when controlling communication through the receiver, receive the first elapsed time in addition to the biological information and the sensor identification information, the first elapsed time being obtained when the biological information is detected.

6. The biological information detection system according to claim 2, wherein the case controller is further configured to:
count a second elapsed time from when it is determined that the biosensor has not been housed in the case, until it is subsequently determined that the biosensor has been housed in the case, and,
as part of the outputting, control the second transmitter to transmit, to the external device, the second elapsed time in addition to the biological information and the sensor identification information in association with one another, the second elapsed time being obtained when it is determined that the biosensor has been housed.

7. The biological information detection system according to claim 2, wherein the case controller is configured to:
count a second elapsed time from when it is determined that the biosensor has not been housed in the case, until it is subsequently determined that the biosensor has been housed in the case, and
output information indicating that the biosensor has not been housed in the case, when the second elapsed time exceeds a predetermined period of time.

8. The biological information detection system according to claim 2,
wherein the biosensor comprises a capacitive sensor configured to detect an electrostatic capacity value as data for calculating the biological information,
wherein the case further comprises a conductor disposed in contact with the capacitive sensor when the biosensor is housed in the case, and
wherein the case controller is configured to determine whether the biosensor has been housed in the case on a basis of the electrostatic capacity value detected by the capacitive sensor.

9. The biological information detection system according to claim 2,
wherein the case further comprises:
a housing space for housing the biosensor, and
a sound emitter configured to emit sound toward the housing space,
wherein the biosensor further comprises:
a sound collector configured to detect sound emitted by a living body, and
a sensor controller configured to determine whether the biosensor has been housed in the case,
wherein, when the sound collector detects a first sound based on the sound emitted by the sound emitter, the sensor controller is configured to control transmission of a first signal through the first transmitter, and
wherein, when the first signal has been received through the receiver, the case controller is configured to determine that the biosensor has been housed in the case.

10. The biological information detection system according to claim 9,
wherein the biosensor comprises:
a cylindrical body, and
a diaphragm that is detachable from an opening of the body,
wherein, when the sound collector detects a second sound, the sensor controller is configured to control transmission of a second signal through the first transmitter, the second sound being determined in advance as a sound different from the first sound, and
wherein, when the second signal has been received through the receiver, the case controller is configured to determine that there is an abnormality in the diaphragm.

11. The biological information detection system according to claim 10, wherein, when it is determined that there is an abnormality in the diaphragm, the case controller is configured to, as part of the outputting, control communication through the first transmitter to transmit, to the external device, information indicating that there is an abnormality in the diaphragm.
